(19) United States
(12) Patent Application Publication
(10) Pub. No.: US 2026/0142052 A1
(43) Pub. Date: May 21, 2026

(54) FIRE IMPACT MINIMIZATION SYSTEM OF SPENT NUCLEAR FUEL STORAGE FACILITY

(71) Applicant: KEPCO ENGINEERING & CONSTRUCTION COMPANY, INC., Gimcheon-si (KR)

(72) Inventors: Dong Jin LEE, Gimcheon-si (KR); Kwang Soon CHOI, Daejeon (KR); You Soo KIM, Gimcheon-si (KR); Hyung Jun YOON, Gimcheon-si (KR); Kyung Tae KIM, Gimcheon-si (KR)

(21) Appl. No.: 19/366,669

(22) Filed: Oct. 23, 2025

(30) Foreign Application Priority Data
Nov. 18, 2024 (KR) 10-2024-0163980

(51) Int. Cl. G21F 9/00; A62C 3/00; G01N 33/28; G21F 7/06; G21F 9/22; G21F 9/34

(52) U.S. Cl. CPC G21F 9/008; A62C 3/002; G01N 33/28; G21F 7/063; G21F 9/22; G21F 9/34

(57) ABSTRACT

A fire impact minimization system of a spent nuclear fuel storage facility includes a storage in which a nuclear fuel storage vessel is safely placed, a trench provided outside the storage and having a space for collecting aviation fuel, an oil discharge hole provided in the trench for discharging aviation fuel, and a drainage pipe connected to the oil discharge hole and configured to discharge the aviation fuel to an outside of the trench.

### CROSS-REFERENCE TO RELATED APPLICATION

This application is based on and claims priority under 35 U.S.C. § 119 to Korean Patent Application No. 10-2024-0163980, filed on Nov. 18, 2024, in the Korean Intellectual Property Office, the disclosure of which is incorporated by reference herein in its entirety.

### BACKGROUND

### 1. Field

This work was supported by the Korea Institute of Energy Technology Evaluation and Planning(KETEP) and the Ministry of Trade, Industry & Energy(MOTIE) of the Republic of Korea (No. RS-2021-KP002663).

The present disclosure relates to a fire impact minimization system of a spent nuclear fuel storage facility, and more specifically, to a fire impact minimization system of a spent nuclear fuel storage facility, which may minimize the possibility of fire and explosion caused by aviation fuel by quickly discharging aviation fuel that may flow into a storage facility in the event of an aircraft crash.

### 2. Description of the Related Art

Recently, there has been a growing demand for safety measures in the conceptual design of a spent nuclear fuel management facility. Also, a spent nuclear fuel storage facility using in-building container storage method has to be designed such that safety functions of the facility are not impaired by the impact of foreseeable external man-made events, such as aircraft crashes in the event of terrorist acts.

Many airports and air routes for military and medium-and-large-sized commercial aircrafts are located near urban areas or key national facilities. With a large number of aircraft in operation, there is a potential risk of terrorist attacks, such as aircraft crashes in spent nuclear fuel storage facilities.

When an aircraft collides with a spent nuclear fuel storage facility, a large amount of aviation fuel from the aircraft may flow into the spent nuclear fuel storage facility. Aviation fuel flowing into the spent nuclear fuel storage facility may cause a fire or explosion in the spent nuclear fuel storage facility, which may result in a disaster, such as a radiation leak.

However, general spent nuclear fuel storage facilities lack a design concerning an aviation fuel drainage system that may discharge aviation fuel in the event of an aircraft crash.

While general spent nuclear fuel storage facilities are designed for collecting wastewater generated within the building, this design alone makes it impossible to separate wastewater and aviation fuel. Also, a general drainage system design of a general spent nuclear fuel storage facility makes it difficult to quickly discharge flammable aviation fuel to prevent fire and explosion.

Therefore, a design for an aviation fuel drainage system for rapidly discharging aviation fuel that may potentially flow into a spent nuclear fuel storage facility in the event of an aircraft crash is required to reduce the impact of fire and explosion.

### SUMMARY

The present disclosure provides a fire impact minimization system of a spent nuclear fuel storage facility, which may reduce the possibility of fire and explosion caused by aviation fuel by quickly discharging aviation fuel that may flow into the storage facility in the event of an aircraft crash.

Additional aspects will be set forth in part in the description which follows and, in part, will be apparent from the description, or may be learned by practice of the presented embodiments of the disclosure.

According to an aspect of the present disclosure, a fire impact minimization system of a spent nuclear fuel storage facility includes a storage in which a nuclear fuel storage vessel is safely placed, a trench provided outside the storage and having a space for collecting aviation fuel, an oil discharge hole provided in the trench for discharging aviation fuel, and a drainage pipe connected to the oil discharge hole and configured to discharge the aviation fuel to an outside of the trench.

The fire impact minimization system may further include a conduit connected to the drainage pipe and buried outside the trench under the spent nuclear fuel storage facility.

The conduit may include a concrete material.

The fire impact minimization system may further include a storage tank connected to the conduit and storing the aviation fuel.

The conduit may include an exhaust pipe communicating with outside air.

The fire impact minimization system may further include a radioactive gas detector provided in the exhaust pipe and configured to detect radioactive gas, a shut-off valve configured to control opening and closing of the exhaust pipe, and a first controller configured to control an operation of the shut-off valve in response to a signal from the radioactive gas detector, wherein the first controller may be further configured to operate the shut-off valve to close the exhaust pipe when the radioactive gas detector detects radioactive gas.

A bottom of the storage may be formed with a downward slope from a center of the storage toward the trench.

The trench may include a curb extending to a preset height, the curb being provided around the oil discharge hole.

The fire impact minimization system may further include an oil content meter provided in the trench and configured to detect oil content, and a second controller configured to control opening and closing of the oil discharge hole in response to a signal detected by the oil content meter, wherein the second controller may be further configured to open the oil discharger hole when oil content is detected by the oil content meter.

The fire impact minimization system may further include a drainage hole provided in the trench and configured to discharge water.

The fire impact minimization system may further include an oil content meter provided in the trench and configured to detect oil content, and a third controller configured to control opening and closing of the oil discharge hole and the drainage hole in response to a signal detected by the oil content meter, wherein the third controller may be further configured to open the oil discharge hole and close the drainage hole when oil content is detected by the oil content meter.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a view illustrating a fire impact minimization system of a spent nuclear fuel storage facility, according to an embodiment;

FIG. 2 is a view illustrating a trench, an oil discharge hole, a discharge pipe, a conduit, an exhaust pipe, and a collection tank according to an embodiment;

FIG. 3 is a view illustrating an exhaust pipe including a radioactive gas detector, a first controller, and a shut-off valve, and a system for controlling opening and closing of the shutoff valve by using the first controller, according to an embodiment.

FIG. 4 is a view illustrating a curb provided around a trench in which an oil discharge hole is arranged, according to an embodiment;

FIG. 5 is a view illustrating an oil content meter provided in a trench and a system for controlling opening and closing of an oil discharge hole by using a second controller, according to an embodiment; and

FIG. 6 is a view illustrating an oil content meter provided in a trench and a system for controlling opening and closing of an oil discharge hole and a drainage hole by using a third controller, according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Following descriptions clarify the scope of the present disclosure, explain principles of the present disclosure and disclose embodiments such that those skilled in the art to which the present disclosure belongs may practice the present disclosure. The disclosed embodiments may be implemented in various forms.

The term “include” or “may include,” which may be used in various embodiments, indicate the presence of the disclosed function, operation, or component, and do not limit one or more additional functions, operations, or components. Also, in various embodiments, the term “comprise”, “include”, or “have” is intended to indicate the presence of a feature, a number, a step, an operation, a component, a portion, or a combination thereof described in the description, and should be understood as not excluding the possibility of the presence or addition of one or more other features, numbers, steps, operations, components, portions, or combinations thereof.

When a component is referred to as being “connected or coupled” to another component, it should be understood that the component may be directly connected or coupled to another component, but that there may also be a new component between the component and another component. In contrast to this, when a component is referred to as being “directly connected” or “directly coupled” to another component, it should be understood that no new component exists between the component and another component.

While the terms “first,” “second”, and so on, used herein may be used to describe various components, the components should not be limited by the terms. Terms are used solely to distinguish one component from another.

The present disclosure relates to a fire impact minimization system of a spent nuclear fuel storage facility, and specifically, to a fire impact minimization system of a spent nuclear fuel storage facility, which may reduce the possibility of fire and explosion caused by aviation fuel by quickly discharging the aviation fuel that may flow into the storage facility in the event of an aircraft crash. Hereinafter, preferred embodiments are described in detail with reference to the accompanying drawings.

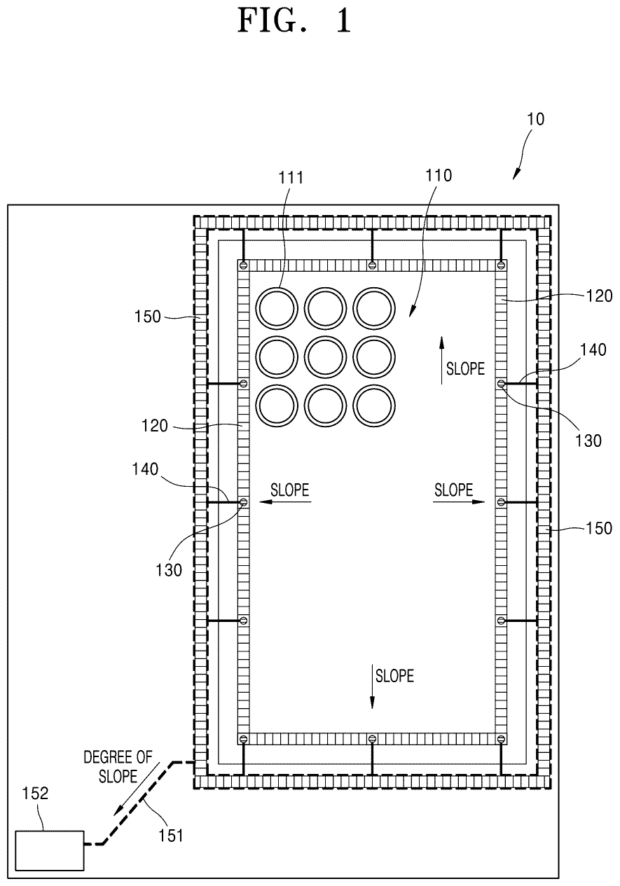

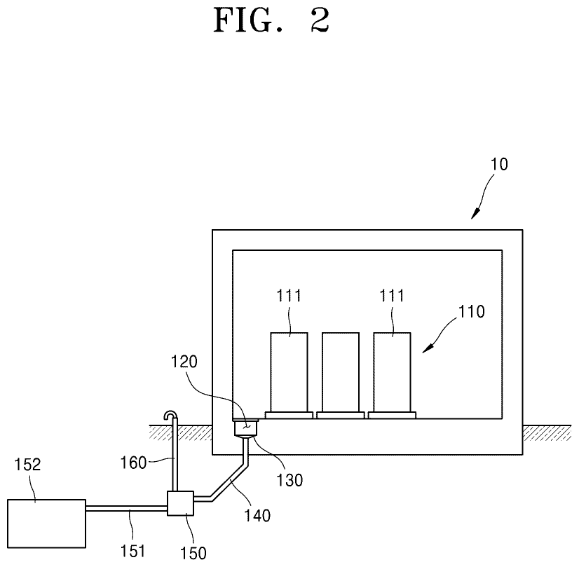

Referring to FIGS. 1 and 2, a fire impact minimization system of a spent nuclear fuel storage facility according to an embodiment includes an aviation fuel discharge system to discharge aviation fuel to a spent nuclear fuel storage facility 10.

Referring to FIGS. 1 and 2, the fire impact minimization system of a spent nuclear fuel storage facility according to an embodiment includes a storage 110, a trench 120, an oil discharge hole 130, and a discharge pipe 140.

A nuclear fuel storage vessel 111 is safely placed in the storage 110. The spent nuclear fuel storage facility 10 may include the storage 110 that is a building structure (wall) on which the nuclear fuel storage vessel 111 is placed. A plurality of nuclear fuel storage vessels 111 may be safely placed in the storage 110.

Referring to FIGS. 1 and 2, the trench 120 may be provided on the outside of the storage 110 and provides a space for collecting aviation fuel. The trench 120 may be provided adjacent to the storage 110 inside a building structure in which the storage 110 is provided.

The trench 120 may surround the outside of the storage 110 and may have a groove shape dug downward. Aviation fuel may be collected in the trench 120 having a groove shape dug downward. Specifically, aviation fuel that may flow into the spent nuclear fuel storage facility 10 in the event of an aircraft crash may be moved to a space inside the trench 120.

The oil discharge hole 130 may be provided in the trench 120 and discharges aviation fuel. The oil discharge hole 130 is provided in the bottom of the trench 120 having a groove shape dug downward, and aviation fuel collected in the trench 120 may be discharged to the outside through the oil discharge hole 130.

The discharge pipe 140 may be connected to the oil discharge hole 130 and discharge aviation fuel to the outside of the trench 120. The discharge pipe 140 may be connected to the oil discharge hole 130, and aviation fuel collected in the trench 120 may pass through the oil discharge hole 130 to move to the discharge pipe 140.

According to an embodiment, the trench 120 may be provided with a plurality of oil discharge holes 130. Also, a plurality of discharge pipes 140 may be connected respectively to the plurality of oil discharge holes 130 of the trench 120.

Referring to FIGS. 1 and 2, the fire impact minimization system of the spent nuclear fuel storage facility according to an embodiment may include a conduit 150 connected to the discharge pipe 140 and buried under the storage facility 10 on the outside of the trench 120.

The conduit 150 may be a pipe buried under the spent nuclear fuel storage facility 10, and may be buried under the spent nuclear fuel storage facility 10 on the outside of a building structure in which the storage 110 and the trench 120 are provided.

According to an embodiment, the conduit 150 may include concrete. The conduit 150 may be a concrete drain including concrete, and may be connected to the discharge pipe 140.

Aviation fuel collected in the trench 120 may move to the discharge pipe 140 through the oil discharge hole 130, and the aviation fuel moved to the discharge pipe 140 may move to the conduit 150.

As described above, a fire impact minimization system of a spent nuclear fuel storage facility according to an embodiment may include the trench 120 inside a building structure in which the storage 110 is provided, and the conduit 150 may be buried under the outside of the building structure, and accordingly, aviation fuel may be quickly discharged.

Referring to FIG. 2, the fire impact minimization system of the spent nuclear fuel storage facility 10 according to an embodiment may include a storage tank 152 in which aviation fuel is stored, and a connection pipe 151 connecting the storage tank 152 to the conduit 150.

The storage tank 152 may be a tank that is connected to the conduit 150 and provided with a space capable of storing aviation fuel. The connection pipe 151 may connect the storage tank 152 to the conduit 150, and aviation fuel transferred to the conduit 150 through the connection pipe 151 may be transferred to the storage tank 152 and stored in the storage tank 152.

In the fire impact minimization system of the spent nuclear fuel storage facility 10 according to an embodiment, a size of the storage tank 152 may be reduced by burying the conduit 150, which is a concrete drain way, outside a building structure in which the storage 110 and the trench 120 are provided.

Because aircraft crashes occur rarely, making the storage tank 152 large enough to store aviation fuel reduces space utilization. However, making the storage tank 152 small may pose a risk of fire and explosion caused by insufficient aviation fuel storage.

In the fire impact minimization system of the spent nuclear fuel storage facility 10 according to an embodiment, the volume capable of storing aviation fuel may be increases by separately providing the conduit 150 buried under the outside of a building structure including the storage 110 and the trench 120, and accordingly, a size of the storage tank 152 may be reduced.

Referring to FIG. 1, a floor of the storage 110 according to an embodiment may have a downward slope from the center of the storage 110 toward the trench 120.

By forming a slight slope in the floor of the storage 110 toward the trench 120, aviation fuel including combustible (flammable) and hazardous materials may be quickly discharged from the storage 110 to the trench 120.

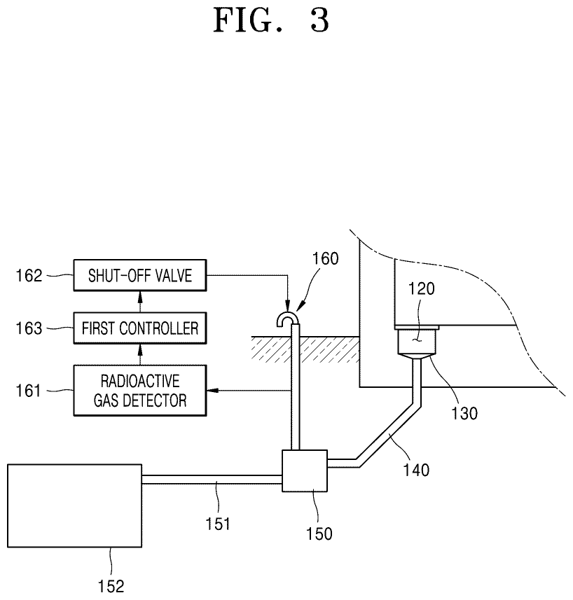

Referring to FIG. 3, the conduit 150 according to an embodiment may include an exhaust pipe 160 communicating with outside air. In general, aviation fuel may include flammable and volatile materials, and accordingly, the aviation fuel may easily vaporize. when aviation fuel vaporizes in the conduit 150, there is a risk that the conduit 150 may be damaged due to an increase in internal pressure of the conduit 150.

To prevent this, the conduit 150 may include the exhaust pipe 160 communicating with outside air. The exhaust pipe 160 may cause the conduit 150 to communicate with outside air, and the gas vaporized in the conduit 150 may be discharged to the outside through the exhaust pipe 160.

By discharging the vaporized gas to the outside through the exhaust pipe 160, the internal pressure of the conduit 150 may be prevented from increasing, and accordingly, the conduit 150 may be prevented from damage due to pressure.

The fire impact minimization system of the spent nuclear fuel storage facility 10 according to an embodiment may further include a radioactive gas detector 161, a shut-off valve 162, and a first controller 163.

Referring to FIG. 3, the radioactive gas detector 161 may be provided in the exhaust pipe 160 to detect radioactive gas. The shut-off valve 162 may control opening and closing of the exhaust pipe 160, and the first controller 163 controls an operation of the shut-off valve 162 in response to a signal from the radioactive gas detector 161.

In the event of an aircraft crash, it is preferable for only aviation fuel to be discharged through the conduit 150, but radioactive materials may also be unintentionally discharged through the conduit 150. When the exhaust pipe 160 is provided in the conduit 150 and a radioactive material leaks to the outside through the exhaust pipe 160, a major disaster may occur.

To prevent such a situation, the fire impact minimization system for the spent nuclear fuel storage facility 10 according to an embodiment may use the radioactive gas detector 161, the shut-off valve 162, and the first controller 163.

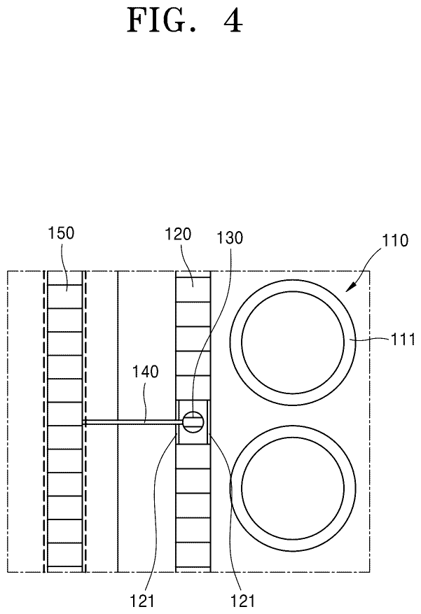

According to an embodiment, when radioactive gas is detected by the radioactive gas detector 161, the first controller 163 may operate the shut-off valve 162 to close the exhaust pipe 160. Accordingly, it is possible to prevent a radioactive material from leaking out through the exhaust pipe 160 Referring to FIG. 4, the trench 120 according to an embodiment may include a curb 121 extending to a preset height, and the curb 121 may be provided around the oil discharge hole 130.

When the nuclear fuel storage vessel 111 falls over the trench 120 due to an aircraft crash, the discharge of aviation fuel may not proceed smoothly. In particular, when the nuclear fuel storage vessel 111 falls over the oil discharge hole 130 of the trench 120 due to an aircraft crash, the aviation fuel may not flow smoothly.

To prevent this, the curb 121 may be provided around the oil discharge hole 130. By providing the curb 121 extending to a certain height around the oil discharge hole 130, the oil discharge hole 130 may be prevented from being blocked by the nuclear fuel storage vessel 111. Accordingly, the integrity of a discharge path of the trench 120 may be obtained.

The fire impact minimization system of the spent nuclear fuel storage facility 10 according to an embodiment may include an oil content meter 170 and a second controller 171 that may control opening and closing of the oil discharge hole 130.

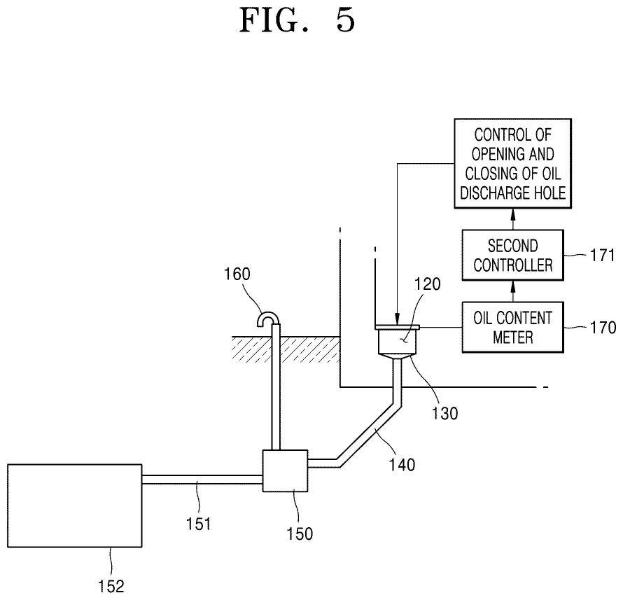

Referring to FIG. 5, the oil content meter 170 may be provided in the trench 120 and may detect oil content. The second controller 171 may control the opening and closing of the oil discharge hole 130, and may control the opening and closing of the oil discharge hole 130 in response to a signal detected by the oil content meter 170.

The spent nuclear fuel storage facility 10 may include a drainage facility that may drain water, and water may flow into the trench 120 through the drainage facility. When the oil discharge hole 130 is constantly open, there is a risk that water flows into the conduit 150.

The conduit 150 may be used for supplying aviation fuel in the event of an aircraft crash, and it is preferable that water does not flow into the conduit 150 during a normal operation, that is, when no aircraft crash occurs.

For this purpose, the trench 120 may include the oil content meter 170 and the second controller 171 that controls the opening and closing of the oil discharge hole 130. Referring to FIG. 5, the second controller 171 may open the oil discharge hole 130 when oil content is detected by the oil content meter 170.

Detecting oil content in the trench 120 may indicate that aviation fuel flows into the trench 120, and when it is determined that aviation fuel flows into the trench 120, the second controller 171 may open the oil discharge hole 130.

Also, when oil is not detected by the oil content meter 170, the second controller 171 may close the oil discharge hole 130 to block water from flowing in.

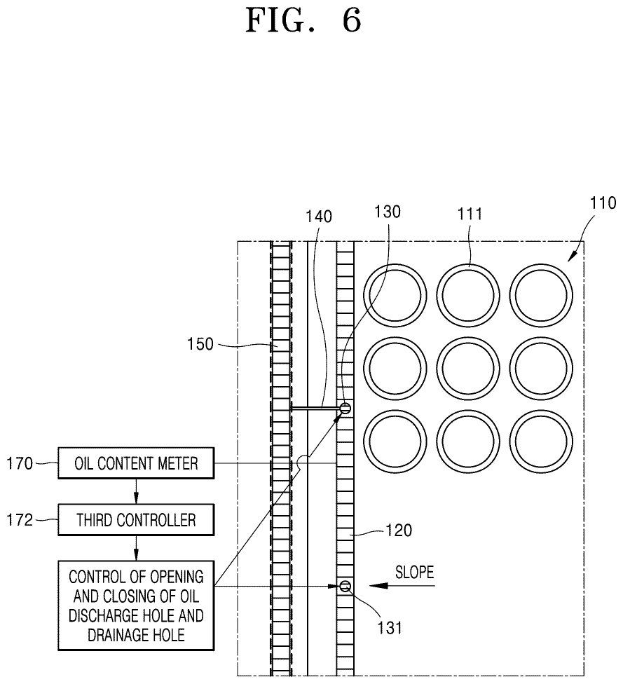

Referring to FIG. 6, a fire impact minimization system of a spent nuclear fuel storage facility according to an embodiment may include a drainage hole 131 that is provided in the trench 120 and discharges water. The drainage hole 131 may be a drainage facility for discharging water, and the trench 120 may also include the drainage hole 131 connected to the drainage facility in addition to the oil discharge hole 130.

The fire impact minimization system of the spent nuclear fuel storage facility according to an embodiment may include a third controller 172 that controls opening and closing of the oil discharge hole 130 and the drainage hole 131 in response to a signal detected by the oil content meter 170.

Aviation fuel, which is a combustible material, is composed of kerosene and has a specific gravity of about 0.775 to about 0.840. The spent nuclear fuel storage facility 10 may include a canister which seal a spent nuclear fuel assembly and maintain a containment function, and a passive cooling system that removes heat from a storage vessel.

In the event of an aircraft crash, aviation fuel may flow into the facility through a local damaged portion and a passive cooling air inlet. In this case, it is preferable to separately discharge aviation fuel (oil) and floor drain (water) to reduce the impact of fire and explosion.

To this end, the trench 120 may include the oil content meter 170, and the third controller 172 that controls opening and closing of the oil discharge hole 130 and the drainage hole 131.

When aviation fuel flows into the trench 120, oil content may be detected by the oil content meter 170, and the third controller 172 may control opening and closing of the oil discharge hole 130 and the drainage hole 131 in response to a signal detected by the oil content meter 170.

Specifically, referring to FIG. 6, when oil content is detected by the oil content meter 170, the third controller 172 may cause the oil discharge hole 130 to be opened and cause the drainage hole 131 to be closed.

In this way, as the third controller 172 controls the opening and closing of the oil discharge hole 130 and the drainage hole 131, only aviation fuel may be separately discharged, and thereby, it is possible to prevent the water generated during a normal operation of the spent nuclear fuel storage facility 10 from flowing into the conduit 150.

The fire impact minimization system of the spent nuclear fuel storage facility according to the embodiment described above has following effects.

The fire impact minimization system of the spent nuclear fuel storage facility according to the embodiment has an advantage of reducing the possibility of fire and explosion caused by aviation fuel by quickly discharging the aviation fuel that may flow into a storage facility in the event of an aircraft crash.

Also, the fire impact minimization system of a spent nuclear fuel storage facility according to the embodiment may reduce the possibility of fire and explosion and a duration of the fire and explosion by quickly discharging aviation fuel through a trench having a space in which the aviation fuel is collected, an oil discharge hole for discharging the aviation fuel, and a concrete conduit connected to the oil discharge hole.

Also, the fire impact minimization system of the spent nuclear fuel storage facility according to the embodiment has an advantage of reducing the impact of fire and explosion on a structures, a system, and equipment of the spent nuclear fuel storage facility by reducing the possibility of fire and explosion and a duration of the fire and explosion.

Although the present disclosure is described above with reference to the embodiments illustrated in the drawings, these are merely examples, and those skilled in the art will appreciate that various modifications and changes of the embodiments may be made. Therefore, the true scope of protection of the present disclosure should be defined by the technical idea of the appended claims.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A fire impact minimization system of a spent nuclear fuel storage facility in an event of an aircraft crash, the fire impact minimization system comprising:
a storage in which a nuclear fuel storage vessel is safely placed;
a trench provided outside the storage and having a space for collecting aviation fuel;
an oil discharge hole provided in the trench for discharging aviation fuel; and
a drainage pipe connected to the oil discharge hole and configured to discharge the aviation fuel to an outside of the trench.

2. The fire impact minimization system of claim 1, further comprising a conduit connected to the drainage pipe and buried outside the trench under the spent nuclear fuel storage facility.

3. The fire impact minimization system of claim 2, wherein the conduit includes a concrete material.

4. The fire impact minimization system of claim 2, further comprising a storage tank connected to the conduit and storing the aviation fuel.

5. The fire impact minimization system of claim 2, wherein the conduit includes an exhaust pipe communicating with outside air.

6. The fire impact minimization system of claim 5, further comprising:
a radioactive gas detector provided in the exhaust pipe and configured to detect radioactive gas;
a shut-off valve configured to control opening and closing of the exhaust pipe; and
a first controller configured to control an operation of the shut-off valve in response to a signal from the radioactive gas detector,
wherein the first controller is further configured to operate the shut-off valve to close the exhaust pipe when the radioactive gas detector detects radioactive gas.

7. The fire impact minimization system of claim 1, wherein a bottom of the storage is formed with a downward slope from a center of the storage toward the trench.

8. The fire impact minimization system of claim 1, wherein
the trench includes a curb extending to a preset height, and
the curb is provided around the oil discharge hole.

9. The fire impact minimization system of claim 1, further comprising:
an oil content meter provided in the trench and configured to detect oil content; and
a second controller configured to control opening and closing of the oil discharge hole in response to a signal detected by the oil content meter,
wherein the second controller is further configured to open the oil discharger hole when oil content is detected by the oil content meter.

10. The fire impact minimization system of claim 1, further comprising a drainage hole provided in the trench and configured to discharge water.

11. The fire impact minimization system of claim 10, further comprising:
an oil content meter provided in the trench and configured to detect oil content; and
a third controller configured to control opening and closing of the oil discharge hole and the drainage hole in response to a signal detected by the oil content meter,
wherein the third controller is further configured to open the oil discharge hole and close the drainage hole when oil content is detected by the oil content meter.
